(19) United States
(12) Patent Application Publication
(10) Pub. No.: US 2025/0144087 A1
(43) Pub. Date: May 8, 2025

(54) USE OF VE607 FOR THE TREATMENT OF SARS-COV-2 INFECTIONS AND RELATED DISEASES

(71) Applicants: Andrés FINZI, Quebec (CA); Marzena PAZGIER, Derwood, MD (US); Amos B. SMITH, III, Merion Station, PA (US); Shilei DING, Quebec (CA); Walter MOTHES, Middletown, CT (US); Christian BARON, Quebec (CA); Derek YANG, Sunnyvale, CA (US); Mohammadjavad MOHAMMADI, Philadelphia, PA (US); Cameron ABRAMS, Philadelphia, PA (US)

(72) Inventors: Andrés FINZI, Quebec (CA); Marzena PAZGIER, Derwood, MD (US); Amos B. SMITH, III, Merion Station, PA (US); Shilei DING, Quebec (CA); Walter MOTHES, Middletown, CT (US); Christian BARON, Quebec (CA); Derek YANG, Sunnyvale, CA (US); Mohammadjavad MOHAMMADI, Philadelphia, PA (US); Cameron ABRAMS, Philadelphia, PA (US)

(21) Appl. No.: 18/835,301

(22) PCT Filed: Jan. 31, 2023
(86) PCT No.: PCT/CA2023/050130

§ 371 Date: Aug. 1, 2024

Related U.S. Application Data
(60) Provisional application No. 63/267,409, filed on Feb. 1, 2022.

(51) Int. Cl. A61K 31/4545; A61K 45/06; A61P 31/14

(52) U.S. Cl. CPC A61K 31/4545; A61K 45/06; A61P 31/14

(57) ABSTRACT

The present application relates to the use of 3,3′-(1,3-phenylenebis(oxy))bis(1-(piperidin-1-yl)propan-2-ol (VE607) or a pharmaceutically acceptable salt thereof for the inhibition of SARS-CoV-2 and/or management of COVID-19.

### CROSS REFERENCE TO RELATED APPLICATIONS

The present application claims the benefit of U.S. provisional patent application No. 63/267,409, filed on Feb. 1, 2022, which is incorporated herein by reference in its entirety.

### TECHNICAL FIELD

The present disclosure generally relates to viral infection, and more particularly to the prevention and/or treatment of coronavirus infections such as severe acute respiratory syndrome coronavirus 2 (SARS-CoV-2) infection.

### BACKGROUND ART

Coronaviruses are large, roughly spherical, RNA viruses with bulbous surface projections that cause diseases in mammals and birds. In humans, these viruses cause respiratory tract infections that can range from mild to lethal. Mild illnesses include some cases of the common cold (which is also caused by other viruses, predominantly rhinoviruses), while more lethal varieties can cause severe acute respiratory syndrome (SARS), Middle East respiratory syndrome (MERS), and Coronavirus disease 2019 (COVID-19). Coronaviruses have four structural proteins, namely the Spike (S), Envelope (E), and Membrane (M) proteins, forming the viral envelope, as well as the Nucleocapsid (N) protein, holding the viral RNA genome.

Severe acute respiratory syndrome coronavirus 2 (SARS-CoV-2) is the strain of coronavirus that causes COVID-19, the respiratory illness responsible for the COVID-19 pandemic. The spike protein of SARS-CoV-2 is the glycoprotein responsible for allowing the virus to attach to and fuse with the membrane of a host cell; specifically, its S1 subunit contains the receptor-binding domain (RBD) that interacts with the cellular receptor angiotensin-converting enzyme 2 (ACE2) enabling virus attachment, and its S2 subunit possesses the fusion machinery, which can mediate host-viral membrane fusion after S1 shedding. The main receptor involved in SARS-CoV-2 entry into human cells is the angiotensin converting enzyme 2 (ACE2). After attachment of a SARS-CoV-2 virion to a target cell, the cell's protease transmembrane protease, serine 2 (TMPRSS2) cuts open the spike protein of the virus, exposing a fusion peptide in the S2 subunit, enabling viral fusion.

Multiple variants of SARS-CoV-2 are circulating globally and within the United States. New variants that have rapidly become dominant within their countries have aroused concerns: B.1.1.7 (also known as VOC-202012/01 or Alpha), 501Y.V2 (B.1.351 or Beta), P.1 (B.1.1.28.1 or Gamma), Delta (B.1.617.2) and B.1.1.529 (Omicron, which includes BA.1, BA.2 and BA.3 sublineages).

The B.1.1.7 variant (23 mutations with 17 amino acid changes) was first described in the United Kingdom in December 2020; the 501Y.V2 variant (23 mutations with 17 amino acid changes) was initially reported in South Africa in December 2020; and the P.1 variant (approximately 35 mutations with 17 amino acid changes) was reported in Brazil in January 2021. By February 2021, the B.1.1.7 variant had been reported in 93 countries, the 501Y.V2 variant in 45, and the P.1 variant in 21. All three variants have the N501Y mutation, which changes the amino acid asparagine (N) to tyrosine (Y) at position 501 in the receptor-binding domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity). Recently, two more SARS-CoV-2 variants, B.1.427 and B.1.429, which were first detected in California, have been shown to be approximately 20% more transmissible than pre-existing variants and have been classified by the CDC as variants of concern. The B.1.617.2 Delta variant comprises the following substitutions in the Spike protein that are known to affect transmissibility of the virus: D614G, T478K, P681R and L452R. The B.1.1.529 (Omicron) variant was reported to the WHO in November 2021 and comprises 32 mutations in the Spike protein. Studies on these variants have provided compelling evidence that they have the potential to escape naturally-induced immunity as well as the immunity induced by currently approved vaccines. Indeed, recent evidence suggests that dual-Ab cocktails from Regeneron (casirivimab and imdevimab, targeting adjacent, non-overlapping epitopes) and Eli Lilly (bamlanivimab and etesevimab, targeting overlapping epitopes) are much less effective at neutralizing Omicron (Wilhelm et al., eBioMedicine, vol. 82, 104158 (2022); Gruell et al., Nature Medicine volume 28, pages 477-480 (2022)), and that current vaccines confer a reduced protection against infections by the Omicron variant (Buchan et al., JAMA Netw Open. 2022; 5(9): e2232760).

Thus, there is a need for the development of therapies against SARS-CoV-2, including SARS-CoV-2 variants, and that minimize the risk of viral escape.

The present description refers to a number of documents, the content of which is herein incorporated by reference in their entirety.

### SUMMARY

The present disclosure provides the following items 1 to 36:
1. A method for preventing or treating a SARS-CoV-2 infection or a related disease in a subject in need thereof, the method comprising administering to the subject an effective amount of 3,3′-(1,3-phenylenebis(oxy))bis(1-(piperidin-1-yl)propan-2-ol (VE607) or a pharmaceutically acceptable salt thereof.
2. A method for reducing the risk of developing a SARS-CoV-2-related disease or the severity of a SARS-CoV-2-related disease in a subject, the method comprising administering to the subject an effective amount of 3,3′-(1,3-phenylenebis(oxy))bis(1-(piperidin-1-yl)propan-2-ol (VE607) or a pharmaceutically acceptable salt thereof.
3. A method (in vitro or in vivo) for blocking the entry of SARS-CoV-2 in an ACE2-expressing cell, the method comprising contacting the SARS-CoV-2 and/or cell with an effective amount of 3,3′-(1,3-phenylenebis(oxy))bis(1-(piperidin-1-yl)propan-2-ol (VE607) or a pharmaceutically acceptable salt thereof.
4. The method of any one of items 1-3, wherein the SARS-CoV-2 is a SARS-CoV-2 variant.
5. The method of item 4, wherein the SARS-CoV-2 variant comprises one or more of the following mutations in the Spike protein: L5F, S13I, L18F, T19R, T20N, P26S, A67V, del69-70, G75V, T76I, D80Y, D80A, T95I, S98F, R102I, D138Y, G142D, del142-144, del144, W152C, E154K, EFR156-158G, F157L, R190S, ins214EPE, D215G, A222V, del246-252, D253G, W258L, N354D, F342L, V367F, K417N, K417T, A435S, W436R, N439K, N440K, G446V, L452R, Y453F, K458R, G476S, S477N, S477G, T478K, V483A, E484K, E484Q, F490S, N501Y, N501S, N501T, A570D, Q613H, D614G, A626S, A653V, H655Y, Q677H, Q677P, P681H, P681R, A701V, T716I, D796H, D796Y, T859N, F888L, D950N, S982A, T1027I, Q1071H, E1092K, H1101Y, D1118H, V1176F, G1219V, and V1122L.
6. The method of item 4 or 5, wherein the SARS-CoV-2 variant is the Alpha, Beta, Gamma, Delta or Omicron variant.
7. The method of any one of items 1 to 6, wherein the VE607 or pharmaceutically acceptable salt thereof is present in a pharmaceutical composition comprising a pharmaceutically acceptable carrier or excipient.
8. The method of item 7, wherein the pharmaceutical composition is an oral composition.
9. The method of item 8, wherein the pharmaceutical composition is a tablet or a capsule.
10. The method of any one of items 1-9, wherein the VE607 or pharmaceutically acceptable salt thereof is administered in combination with an additional active agent.
11. The method of item 10, wherein the additional active agent is a pain killer, an anti-nausea agent, an anti-coagulant, an anti-inflammatory agent, an immunotherapeutic agent, a SARS-CoV-2 vaccine or a SARS-CoV-2 inhibitor (e.g., an anti-SARS-CoV-2 antibody).
12. The method of any one of items 1-11, wherein the subject is an immunosuppressed or immunocompromised subject.
13. 3,3′-(1,3-phenylenebis(oxy))bis(1-(piperidin-1-yl)propan-2-ol (VE607) or a pharmaceutically acceptable salt thereof for use in preventing or treating a SARS-CoV-2 infection or a related disease in a subject in need thereof.
14. 3,3′-(1,3-phenylenebis(oxy))bis(1-(piperidin-1-yl)propan-2-ol (VE607) or a pharmaceutically acceptable salt thereof for use in reducing the risk of developing a SARS-CoV-2-related disease or the severity of a SARS-CoV-2-related disease in a subject.
15. 3,3′-(1,3-phenylenebis(oxy))bis(1-(piperidin-1-yl)propan-2-ol (VE607) or a pharmaceutically acceptable salt thereof for use in blocking the entry of SARS-CoV-2 in an ACE2-expressing cell in vitro or in vivo.
16. The VE607 or a pharmaceutically acceptable salt thereof for use of any one of items 13-15, wherein the SARS-CoV-2 is a SARS-CoV-2 variant.
17. The VE607 or a pharmaceutically acceptable salt thereof for use of item 16, wherein the SARS-CoV-2 variant comprises one or more of the following mutations in the Spike protein: L5F, S13I, L18F, T19R, T20N, P26S, A67V, del69-70, G75V, T76I, D80Y, D80A, T95I, S98F, R102I, D138Y, G142D, del142-144, del144, W152C, E154K, EFR156-158G, F157L, R190S, ins214EPE, D215G, A222V, del246-252, D253G, W258L, N354D, F342L, V367F, K417N, K417T, A435S, W436R, N439K, N440K, G446V, L452R, Y453F, K458R, G476S, S477N, S477G, T478K, V483A, E484K, E484Q, F490S, N501Y, N501S, N501T, A570D, Q613H, D614G, A626S, A653V, H655Y, Q677H, Q677P, P681H, P681R, A701V, T716I, D796H, D796Y, T859N, F888L, D950N, S982A, T1027I, Q1071H, E1092K, H1101Y, D1118H, V1176F, G1219V, and V1122L.
18. The VE607 or a pharmaceutically acceptable salt thereof for use of item 16 or 17, wherein the SARS-CoV-2 variant is the Alpha, Beta, Gamma, Delta or Omicron variant.
19. The VE607 or a pharmaceutically acceptable salt thereof for use of any one of items 13 to 18, wherein the VE607 or pharmaceutically acceptable salt thereof is present in a pharmaceutical composition comprising a pharmaceutically acceptable carrier or excipient.
20. The VE607 or a pharmaceutically acceptable salt thereof for use of item 19, wherein the pharmaceutical composition is an oral composition.
21. The VE607 or a pharmaceutically acceptable salt thereof for use of item 20, wherein the pharmaceutical composition is a tablet or a capsule.
22. The VE607 or a pharmaceutically acceptable salt thereof for use of any one of items 13-21, wherein the VE607 or pharmaceutically acceptable salt thereof is for administration in combination with an additional active agent.
23. The VE607 or a pharmaceutically acceptable salt thereof for use of item 22, wherein the additional active agent is a pain killer, an anti-nausea agent, an anti-coagulant, an anti-inflammatory agent, an immunotherapeutic agent, a SARS-CoV-2 vaccine or a SARS-CoV-2 inhibitor (e.g., an anti-SARS-CoV-2 antibody).
24. The VE607 or a pharmaceutically acceptable salt thereof for use of any one of items 13-23, wherein the subject is an immunosuppressed or immunocompromised subject.
25. Use of 3,3′-(1,3-phenylenebis(oxy))bis(1-(piperidin-1-yl)propan-2-ol (VE607) or a pharmaceutically acceptable salt thereof for the manufacture of a medicament for preventing or treating a SARS-CoV-2 infection or a related disease in a subject in need thereof.
26. Use of 3,3′-(1,3-phenylenebis(oxy))bis(1-(piperidin-1-yl)propan-2-ol (VE607) or a pharmaceutically acceptable salt thereof for the manufacture of a medicament for reducing the risk of developing a SARS-CoV-2-related disease or the severity of a SARS-CoV-2-related disease in a subject.
27. Use of 3,3′-(1,3-phenylenebis(oxy))bis(1-(piperidin-1-yl)propan-2-ol (VE607) or a pharmaceutically acceptable salt thereof for the manufacture of a medicament for blocking the entry of SARS-CoV-2 in an ACE2-expressing cell in vitro or in vivo.
28. The use of any one of items 25-27, wherein the SARS-CoV-2 is a SARS-CoV-2 variant.
29. The use of item 28, wherein the SARS-CoV-2 variant comprises one or more of the following mutations in the Spike protein: L5F, S13I, L18F, T19R, T20N, P26S, A67V, del69-70, G75V, T76I, D80Y, D80A, T95I, S98F, R102I, D138Y, G142D, del142-144, del144, W152C, E154K, EFR156-158G, F157L, R190S, ins214EPE, D215G, A222V, del246-252, D253G, W258L, N354D, F342L, V367F, K417N, K417T, A435S, W436R, N439K, N440K, G446V, L452R, Y453F, K458R, G476S, S477N, S477G, T478K, V483A, E484K, E484Q, F490S, N501Y, N501S, N501T, A570D, Q613H, D614G, A626S, A653V, H655Y, Q677H, Q677P, P681H, P681R, A701V, T716I, D796H, D796Y, T859N, F888L, D950N, S982A, T1027I, Q1071H, E1092K, H1101Y, D1118H, V1176F, G1219V, and V1122L.
30. The use of item 28 or 29, wherein the SARS-CoV-2 variant is the Alpha, Beta, Gamma, Delta or Omicron variant.
31. The use of any one of items 25-30, wherein the VE607 or pharmaceutically acceptable salt thereof is present in a pharmaceutical composition comprising a pharmaceutically acceptable carrier or excipient.
32. The use of item 31, wherein the pharmaceutical composition is an oral composition.
33. The use of item 32, wherein the pharmaceutical composition is a tablet or a capsule.
34. The use of any one of items 25-33, wherein the VE607 or pharmaceutically acceptable salt thereof is for administration in combination with an additional active agent.
35. The use of item 34, wherein the additional active agent is a pain killer, an anti-nausea agent, an anti-coagulant, an anti-inflammatory agent, an immunotherapeutic agent, a SARS-CoV-2 vaccine or a SARS-CoV-2 inhibitor (e.g., an anti-SARS-CoV-2 antibody).
36. The use of any one of items 25-35, wherein the subject is an immunosuppressed or immunocompromised subject.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1A depicts the chemical structures of VE607 and SSAA09E2;

FIG. 1B depicts a graph showing the results from two experiments (eight replicates total) of differential scanning fluorimetry of the SARS-CoV-2 RBD in the presence of SARS-CoV-1 inhibitors;

FIGS. 1C and 1D depict the virtual docking of VE607 to SARS-CoV-1 and SARS-CoV-2 RBD, respectively;

FIG. 1E is a graph showing that the stereochemical isomers of VE607 had the same capacity as commercially available VE607 to neutralize SARS-CoV-2 pseudovirus particles. Two stereochemical isomers of VE607, (S,S)-VE607 and (R,R)-VE607, were tested for their inhibition of VSV-G pseudovirus or SARS-CoV-2 D614G pseudovirus particles. IC₅₀ values are shown next to the different VOC Spikes. Data represents the average of at least four independent experiments +/−SEM.

FIG. 2A is a graph showing the VE607 inhibition of SARS-CoV-1, SARS-CoV-2 or VSV-G (specificity control) pseudovirus;

FIG. 2B is a graph showing the VE607 inhibition of authentic live SARS-CoV-2;

FIG. 2C is a graph showing that VE607 is not toxic on VERO-E6 cells, as measured by AquaVivid staining;

FIG. 2D is a graph showing pseudovirus neutralization of SARS-CoV-2 S mutants predicted by in silico analysis to modulate the inhibition by VE607;

FIG. 3A is a graph showing that VE607 doesn't compete for sACE2-AF594 interaction, as measured by flow cytometry. The values represent the median fluorescence intensities (MFI) normalized to binding signals obtained with the conformational independent CV3-25 Ab binding;

FIG. 3B is a graph showing SARS-CoV-2 Spike stability as measured by radioactive labelling of 293T Spike expressing cells followed by immunoprecipitation of cell lysates and supernatants. At least 4 experiments are represented as mean±SEM and statistical significance was tested using unpaired t test, * p<0.05;

FIGS. 3C-E are graphs showing the results of single molecule FRET analysis of SARS-CoV-2 S unliganded (FIG. 3C), in presence of ACE2 (FIG. 3D) or VE607 (FIG. 3E);

FIG. 4A is a graph showing that VE607 inhibits the infection of 293T-hACE2 cells by pseudoviral particles expressing the S protein from SARS-CoV-2 variant Alpha, Beta, Gamma, Delta and Omicron (BA.1 sublineage). IC₅₀ values are shown next to the different VOC Spikes. Data represents the average of at least four independent experiments+/−SEM;

FIG. 4B is a graph showing that VE607 inhibits the infection of 293T-hACE2 cells by pseudoviral particles expressing the S protein from SARS-CoV-2 variant Omicron of the BA.1, BA.4/5, BA.4.6, BQ.1.1 or XBB sublineage. IC₅₀ values are shown in the below the graph. Data represents the average of at least two independent experiments+/−SEM;

FIG. 5 is a graph depicting the mutations in the Spike protein from SARS-CoV-2 VOCs Omicron sublineages BA.1, BA.2 and BA.3 (from William A. Haseltine, Birth of The Omicron Family: BA.1, BA.2, BA.3. Each As Different As Alpha Is From Delta, Forbes, Jan. 26, 2022).

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in virology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies reported herein, the present inventors have shown that VE607 has the ability to inhibit or neutralize SARS-CoV-2, including SARS-CoV-2 variants of concern (VOC) such as Delta and Omicron.

The present disclosure provides a method for preventing or treating a SARS-CoV-2 infection or a related disease in a subject in need thereof, the method comprising administering to the subject an effective amount of VE607 or a pharmaceutically acceptable salt thereof. The present disclosure provides the use of VE607 or a pharmaceutically acceptable salt thereof for preventing or treating a SARS-CoV-2 infection or a related disease in a subject. The present disclosure provides the use of VE607 or a pharmaceutically acceptable salt thereof for the manufacture of a medicament for preventing or treating a SARS-CoV-2 infection or a related disease in a subject. The present disclosure provides VE607 or a pharmaceutically acceptable salt thereof for use in preventing or treating a SARS-CoV-2 infection or a related disease in a subject.

The present disclosure also provides a method for reducing the risk of developing a SARS-CoV-2-related disease or the severity of a SARS-CoV-2-related disease in a subject, the method comprising administering to the subject an effective amount of VE607 or a pharmaceutically acceptable salt thereof. The present disclosure provides the use of VE607 or a pharmaceutically acceptable salt thereof for reducing the risk of developing a SARS-CoV-2-related disease or the severity of a SARS-CoV-2-related disease in a subject. The present disclosure provides the use of VE607 or a pharmaceutically acceptable salt thereof for the manufacture of a medicament for reducing the risk of developing a SARS-CoV-2-related disease or the severity of a SARS-CoV-2-related disease in a subject. The present disclosure provides VE607 or a pharmaceutically acceptable salt thereof for use in reducing the risk of developing a SARS-CoV-2-related disease or the severity of a SARS-CoV-2-related disease in a subject.

The present disclosure also provides a method (in vitro or in vivo) for blocking the entry of SARS-CoV-2 in an ACE2-expressing cell, the method comprising contacting the SARS-CoV-2 and/or cell with an effective amount of VE607 or a pharmaceutically acceptable salt thereof. The present disclosure provides the use of VE607 or a pharmaceutically acceptable salt thereof for blocking the entry of SARS-CoV-2 in an ACE2-expressing cell. The present disclosure provides the use of VE607 or a pharmaceutically acceptable salt thereof for the manufacture of a medicament for blocking the entry of SARS-CoV-2 in an ACE2-expressing cell. The present disclosure provides VE607 or a pharmaceutically acceptable salt thereof for use in blocking the entry of SARS-CoV-2 in an ACE2-expressing cell.

VE607 (3,3′-(1,3-phenylenebis(oxy))bis(1-(piperidin-1-yl)propan-2-ol) is a compound of the following structure:

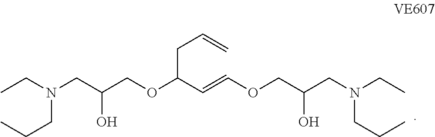 [SMILES: C=CCC(/C=C/OCC(O)CN(CC)CCC)OCC(O)CN(CC)CCC]

The term VE607 as used herein encompasses mixtures (e.g., racemic mixtures) of any one of the enantiomers of VE607, as well as isolated (pure) enantiomers, such as the (S,S)-VE607, (R,R)-VE607, and (R,S)-VE607 (meso) enantiomers.

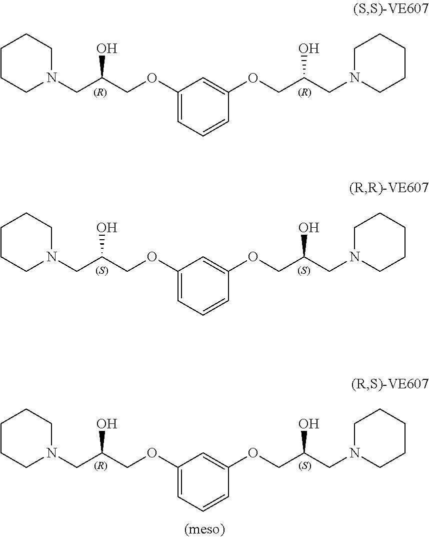

In an embodiment, a racemic mixture is used or administered. In another embodiment, the (R,R)-VE607 enantiomer is used or administered. In another embodiment, the (S,S)-VE607 enantiomer is used or administered. In another embodiment, the (R,S)-VE607 enantiomer is used or administered.

The term “pharmaceutically acceptable salt” refers to salts of VE607 that are pharmacologically acceptable and substantially non-toxic to the subject to which they are administered. More specifically, these salts retain the biological effectiveness and properties of VE607 and are formed from suitable non-toxic organic or inorganic acids or bases.

For example, these salts include acid addition salts of VE607. Such acid addition salts include acetates, adipates, alginates, lower alkanesulfonates such as a methanesulfonates, trifluoromethanesulfonatse or ethanesulfonates, arylsulfonates such as a benzenesulfonates, 2-naphthalenesulfonates, or toluenesulfonates (also known as tosylates), ascorbates, aspartates, benzoates, benzenesulfonates, bisulfates, borates, butyrates, citrates, camphorates, camphorsulfonates, cinnamates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydrogen sulphates, 2-hydroxyethanesulfonates, itaconates, lactates, maleates, mandelates, methanesulfonates, nicotinates, nitrates, oxalates, pamoates, pectinates, perchlorates, persulfates, 3-phenylpropionates, phosphates, picrates, pivalates, propionates, salicylates, succinates, sulfates, sulfonates, tartrates, thiocyanates, undecanoates and the like. In an embodiment, the pharmaceutically acceptable acid salt of VE607 is a hydrochloride salt, including a dihydrochloride salt (3,3′-(1,3-phenylenebis(oxy))bis(1-(piperidin-1-yl)propan-2-ol)dihydrochloride).

VE607 or the pharmaceutically acceptable salt thereof binds to the receptor binding domain (RBD) of SARS-CoV-2 Spike (S) protein and blocks S protein-ACE2-mediated SARS-CoV-2 entry.

The amino acid sequence of the full-length Spike protein from SARS-CoV-2 (Wuhan strain, NCBI Reference Sequence YP_009724390.1, SEQ ID NO: 1) is depicted below:

| | |
|---|---|
| 1 | MFVFLVLLPL VSSQCVNLTT RTQLPPAYTN SFTRGVYYPD KVERSSVLHS TQDLFLPFFS |
| 61 | NVTWFHAIHV SGTNGTKRFD NPVLPFNDGV YFASTEKSNI IRGWIFGTTL DSKTQSLLIV |
| 121 | NNATNVVIKV CEFQFCNDPF LGVYYHKNNK SWMESEFRVY SSANNCTFEY VSQPFLMDLE |
| 181 | GKQGNFKNLR EFVFKNIDGY FKIYSKHTPI NLVRDLPQGF SALEPLVDLP IGINITRFQT |
| 241 | LLALHRSYLT PGDSSSGWTA GAAAYYVGYL QPRTFLLKYN ENGTITDAVD CALDPLSETK |
| 301 | CTLKSFTVEK GIYQTSNFRV QPTESIVRFP NITNLCPFGE VFNATRFASV YAWNRKRISN |
| 361 | CVADYSVLYN SASFSTFKCY GVSPTKLNDL CFTNVYADSF VIRGDEVRQI APGQTGKIAD |
| 421 | YNYKLPDDFT GCVIAWNSNN LDSKVGGNYN YLYRLFRKSN LKPFERDIST EIYQAGSTPC |
| 481 | NGVEGFNCYF PLQSYGFQPT NGVGYQPYRV VVLSFELLHA PATVCGPKKS TNLVKNKCVN |
| 541 | FNFNGLTGTG VLTESNKKFL PFQQFGRDIA DTTDAVRDPQ TLEILDITPC SFGGVSVITP |
| 601 | GTNTSNQVAV LYQDVNCTEV PVAIHADQLT PTWRVYSTGS NVFQTRAGCL IGAEHVNNSY |
| 661 | ECDIPIGAGI CASYQTQTNS PRRARSVASQ SIIAYTMSLG AENSVAYSNN SIAIPTNFTI |
| 721 | SVTTEILPVS MTKTSVDCTM YICGDSTECS NLLLQYGSFC TQLNRALTGI AVEQDKNTQE |
| 781 | VFAQVKQIYK TPPIKDFGGF NESQILPDPS KPSKRSFIED LLFNKVTLAD AGFIKQYGDC |
| 841 | LGDIAARDLI CAQKFNGLTV LPPLLTDEMI AQYTSALLAG TITSGWTFGA GAALQIPFAM |
| 901 | QMAYRFNGIG VTQNVLYENQ KLIANQFNSA IGKIQDSLSS TASALGKLQD VVNQNAQALN |
| 961 | TLVKQLSSNF GAISSVLNDI LSRLDKVEAE VQIDRLITGR LQSLQTYVTQ QLIRAAEIRA |
| 1021 | SANLAATKMS ECVLGQSKRV DFCGKGYHLM SFPQSAPHGV VFLHVTYVPA QEKNFTTAPA |
| 1081 | ICHDGKAHFP REGVFVSNGT HWFVTQRNFY EPQIITTDNT FVSGNCDVVI GIVNNTVYDP |
| 1141 | LQPELDSFKE ELDKYFKNHT SPDVDLGDIS GINASVVNIQ KEIDRLNEVA KNLNESLIDL |
| 1201 | QELGKYEQYI KWPWYIWLGF IAGLIAIVMV TIMLCCMTSC CSCLKGCCSC GSCCKFDEDD |
| 1261 | SEPVLKGVKL HYT |

Residues 1-12 correspond to the signal peptide, residues 13-685 correspond to the Spike protein subunit S1 and residues 686-1273 correspond to the Spike protein subunit S2. The receptor-binding domain (RBD) is defined by residues 319-541 (receptor-binding motif=residues 437-508). Residues 816-837 define the fusion peptide 1, residues 835-855 define the fusion peptide 2, residues 920-970 define the heptad repeat 1 and residues 1163-1202 define the heptad repeat 2.

SARS-CoV2 variants comprise mutations in the Spike protein including L5F, S13I, L18F, T19R, T20N, P26S, A67V, del69-70, G75V, T76I, D80Y, D80A, T95I, S98F, R102I, D138Y, G142D, del142-144, del144, W152C, E154K, EFR156-158G, F157L, R190S, ins214EPE, D215G, A222V, del246-252, D253G, W258L, N354D, F342L, V367F, K417N, K417T, A435S, W436R, N439K, N440K, G446V, L452R, Y453F, K458R, G476S, S477N, S477G, T478K, V483A, E484K, E484Q, F490S, N501Y, N501S, N501T, A570D, Q613H, D614G, A626S, A653V, H655Y, Q677H, Q677P, P681H, P681R, A701V, T716I, D796H, D796Y, T859N, F888L, D950N, S982A, T1027I, Q1071H, E1092K, H1101Y, D1118H, V1176F, G1219V, and V1122L.

The Delta variant comprises the following Spike protein mutations: T19R, (V70F*), T95I, G142D, E156-, F157-, R158G, (A222V*), (W258L*), (K417N*), L452R, T478K, D614G, P681R, D950N.

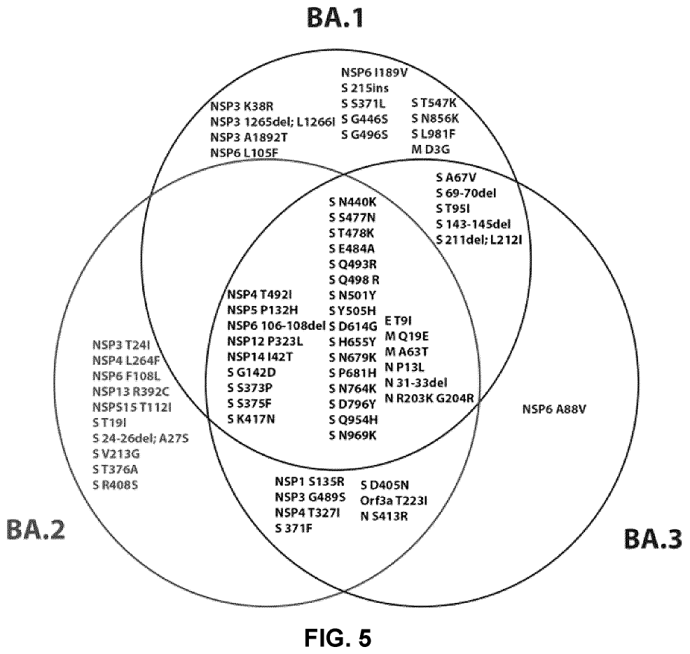

The Omicron variant (sublineage BA.1) comprises the following Spike protein mutations: A67V, del69-70, T95I, del142-144, Y145D, del211, L2121, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F. FIG. 5 depicts the mutations in Omicron sublineages BA.1, BA.2 and BA.3. In an embodiment, the SARS-CoV-2 is of the BA.1, BA.2, BA.3, BA.4/5, BA.4.6, XBB or BQ.1.1 sublineage.

In an embodiment, the methods and uses described herein relates to the inhibition of a SARS-CoV-2 variant comprising one of more of the above-noted mutations, e.g., to the prevention or treatment of diseases caused by such SARS-CoV-2 variant. In an embodiment, the methods and uses described herein relates to the inhibition of a SARS-CoV-2 variant comprising at least 50%, 60%, 70%, 80%, 90% or 95% of the Spike protein mutations of variant Omicron (BA.1, BA.2 and/or BA.3), e.g., to the prevention or treatment of diseases caused by such SARS-CoV-2 variant. In an embodiment, the methods and uses described herein relates to the inhibition of a SARS-CoV-2 variant comprising all of the Spike protein mutations of variant Omicron, e.g., to the prevention or treatment of diseases caused by such SARS-CoV-2 variant.

In another embodiment, VE607 or the pharmaceutically acceptable salt thereof is present in a composition. In an embodiment, the composition further comprises VE607 or the pharmaceutically acceptable salt thereof, and a carrier or excipient, in a further embodiment a pharmaceutically acceptable carrier or excipient. Such compositions may be prepared in a manner well known in the pharmaceutical art by mixing VE607 or the pharmaceutically acceptable salt thereof having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration VE607 or the pharmaceutically acceptable salt thereof by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of VE607 or the pharmaceutically acceptable salt thereof by the oral route.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients (VE607 or the pharmaceutically acceptable salt thereof) and that is not toxic to the subject, i.e., is a type of excipient and/or is for use in an amount which is not toxic to the subject. Excipients are well known in the art, and the present system is not limited in these respects. In certain embodiments, the composition includes excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipient include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness. In an embodiment, VE607 or the pharmaceutically acceptable salt thereof is/are encapsulated in a vesicle or vesicle-like particle, such as a lipid vesicle (e.g., liposome). The term “lipid vesicle” (or “lipid-based vesicle”) as used herein encompasses macromolecular structures which as the main constituent include lipid or lipid derivatives. Suitable examples hereof are liposomes and micelles including detergent micelles/lipid emulsion, liposomes prepared from palmitoyloleoylphosphatidylcholine, hydrogenated soy phosphatdylcholine, and solid lipid nanoparticles prepared from steric acid or tripalmitin. The term liposome is used herein in accordance with its usual meaning, referring to microscopic lipid vesicles composed of a bilayer of phospholipids or any similar amphipathic lipids encapsulating an internal aqueous medium. The liposomes may be unilamellar vesicles such as small unilamellar vesicles (SUVs), which typically have a diameter of less than 0.2 μm (e.g., between 0.02 and 0.2 μm), and large unilamellar vesicles (LUVs), and multilamellar vesicles (MLV), which typically have a diameter greater than 0.45 μm (in some cases greater than 1 μm). No particular limitation is imposed on the liposomal membrane structure in the present disclosure. The term liposomal membrane refers to the bilayer of phospholipids separating the internal aqueous medium from the external aqueous medium. The composition may also comprise one or more additional active agents for the treatment the targeted disease/condition or for the management of symptom(s) of the targeted disease/condition (e.g., pain killers, anti-nausea agents, anti-coagulants, anti-inflammatory agents, immunotherapeutic agents, etc.).

For the prevention, treatment or reduction in the severity of a given disease or condition (viral disease such as COVID-19), the appropriate dosage of VE607 or the pharmaceutically acceptable salt thereof (or pharmaceutical composition described herein) will depend on the type of disease or condition to be treated, the severity and course of the disease or condition, whether VE607 or the pharmaceutically acceptable salt thereof, or pharmaceutical composition, is administered for preventive or therapeutic purposes, previous therapy, the patient's clinical history and response to VE607 or the pharmaceutically acceptable salt thereof, and the discretion of the attending physician. The VE607 or the pharmaceutically acceptable salt thereof may be suitably administered to the patient at one time or over a series of treatments. Preferably, it is desirable to determine the dose-response curve in vitro, and then in useful animal models prior to testing in humans. The present disclosure provides dosages for VE607 or the pharmaceutically acceptable salt thereof, or pharmaceutical composition. For example, depending on the type and severity of the disease, about 1 μg/kg to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

As used herein the term “treating” or “treatment” in reference to viral infection or disease is meant to refer to administration of the agent after infection that leads to a reduction/improvement in one or more symptoms or pathological features associated with said viral disease (e.g., COVID-19). Non-limiting examples include a decrease in viral load, reduction of cough, fever, fatigue, shortness of breath, reduction/prevention of acute respiratory distress syndrome (ARDS), reduction/prevention of multi-organ failure, septic shock, and blood clots, hospitalization, etc.

As used herein the term “preventing” or “prevention” in reference to viral infection or disease is meant to refer to administration of the agent prior to infection that leads to protection from being infected or from developing the viral disease (e.g., COVID-19), to a delay in the development of the disease, or to a reduction of one or more symptoms or pathological features associated with the viral disease.

VE607 or the pharmaceutically acceptable salt thereof, or pharmaceutical composition described herein may be used alone or in combination with other prophylactic agents such as antivirals, anti-inflammatory agents, anti-coagulants, vaccines, immunotherapies (antibody-based therapies), etc. The combination of active agents and/or compositions comprising same may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent (e.g., VE607 or the pharmaceutically acceptable salt thereof) may be administered to a patient before, concomitantly, before and after, or after a second active agent (e.g., an antiviral or anti-inflammatory agent) is administered. The agents may in an embodiment be combined/formulated in a single composition and thus administered at the same time. In another embodiment, VE607 or the pharmaceutically acceptable salt thereof is used in combination with one or anti-SARS-CoV-2 antibodies or antigen-binding fragments thereof (or nucleic acids encoding same), i.e., an antibody cocktail.

In an embodiment, VE607 or the pharmaceutically acceptable salt thereof is for administration prior to exposure to SARS-CoV-2. In another embodiment, VE607 or the pharmaceutically acceptable salt thereof is for administration after exposure to SARS-CoV-2. In another embodiment, VE607 or the pharmaceutically acceptable salt thereof is for administration prior to and after exposure to SARS-CoV-2.

In an embodiment, VE607 or the pharmaceutically acceptable salt thereof is for administration prior to development of the viral disease (e.g., COVID-19). In another embodiment, VE607 or the pharmaceutically acceptable salt thereof is for administration after development of the viral disease (e.g., COVID-19). In another embodiment, VE607 or the pharmaceutically acceptable salt thereof is for administration prior to and after development of the viral disease (e.g., COVID-19).

In an embodiment, the subject or patient has a weakened immune system and a reduced ability to fight viral infections such as SARS-CoV-2 infection. In another embodiment, the subject or patient is an immunosuppressed or immunocompromised subject or patient. Immunosuppression may be caused by certain diseases or conditions, such as AIDS, cancer, diabetes, malnutrition, and certain genetic disorders, or certain drugs or treatments such as anticancer drugs, radiation therapy, and stem cell or organ transplant. In an embodiment, the subject or patient is an elderly subject or patient, for example a subject or patient having 60 years old or more, 65 years old or more, 70 years old or more, 75 years old or more, or 80 years old or more, who typically develop a weaker immune response to vaccines and infections.

In another aspect, the disclosure provides kits for preventing or treating a SARS-CoV-2 infection or a related disease in a subject, or for reducing the risk of developing a SARS-CoV-2-related disease or the severity of a SARS-CoV-2-related disease in a subject, for blocking the entry of SARS-CoV-2 in an ACE2-expressing cell, the kits comprising VE607 or the pharmaceutically acceptable salt thereof. Kits include one or more containers comprising by way of example, and not limitation, VE607 or the pharmaceutically acceptable salt thereof and instructions for use in accordance with any of the methods of the disclosure. The containers can be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The kits are provided in suitable packaging. Suitable packaging includes, but is not limited to, vials, bottles, jars, syringes, flexible packaging (e.g., sealed Mylar or plastic bags), and the like. A kit can have a sterile access port (e.g., the container can be an intravenous solution bag or a vial having a stopper pierceable by a hypodermic injection needle). The container can also have a sterile access port (e.g., the container can be an intravenous solution bag or a vial having a stopper pierceable by a hypodermic injection needle). Kits can optionally provide additional components such as buffers and interpretive information. Normally, the kit comprises a container and a label or package insert(s) on or associated with the container.

### EXAMPLES

The present disclosure is illustrated in further details by the following non-limiting examples.

### Example 1: Materials and Methods

### Plasmids.

The plasmids expressing the human coronavirus Spike D614G of SARS-CoV-2 was kindly provided by Stefan Pöhlmann and was previously reported (Hoffmann et al., 2020b). The pNL4.3 R-E-Luc was obtained from NIH AIDS Reagent Program. The codon-optimized RBD sequence (encoding residues 319-541) fused to a C-terminal hexahistidine tag was cloned into the pcDNA3.1(+) expression vector and was reported elsewhere (Beaudoin-Bussieres et al., 2020). The mutants encoding variants Alpha (B.1.1.7), Beta (B.1.351), Gamma (P.1) were codon-optimized and synthesized by Genscript. Plasmids encoding the Delta and Omicron Spikes were generated by overlapping PCR using a codon-optimized wild-type SARS-CoV-2 Spike gene (GeneArt, ThermoFisher) that was synthesized (Biobasic) and cloned in pCAGGS as a template (Chatterjee et al., 2021; Tauzin et al., 2022). The vesicular stomatitis virus G (VSV-G)-encoding plasmid (pSVCMV-IN-VSV-G) was previously described (Emi et al., 1991).

### Cell Lines.

293T human embryonic kidney cells (obtained from ATCC) and Vero E6 cells (ATCC CRL-1586™) were maintained at 37° C. under 5% CO₂ in Dulbecco's modified Eagle's medium (DMEM) (Wisent) containing 5% fetal bovine serum (VWR), 100 UI/ml of penicillin and 100 μg/ml of streptomycin (Wisent). The 293T-ACE2 cell line was previously reported (Prevost et al., 2020).

### Purification of SARS-CoV-2 RBD.

FreeStyle 293 F cells (Invitrogen) were grown in FreeStyle 293F medium (Invitrogen) to a density of 1×10⁶ cells/mL at 37° C. with 8% CO₂ with regular agitation (150 rpm). Cells were transfected with a plasmid coding for SARS-CoV-2 S RBD using ExpiFectamine 293 transfection reagent, as directed by the manufacturer (Invitrogen). One week later, cells were pelleted and discarded. Supernatants were filtered using a 0.22 μm filter (Thermo Fisher Scientific). The recombinant RBD proteins were purified by nickel affinity columns, as directed by the manufacturer (Invitrogen). The RBD preparations were dialyzed against phosphate-buffered saline (PBS) and stored in aliquots at −80° C. until further use. To assess purity, recombinant proteins were loaded on SDS-PAGE gels and stained with Coomassie Blue.

### Differential Scanning Fluorimetry.

DSF experiments were essentially performed as described previously (Sharifahmadian et al., 2017). DSF was conducted using 5 μM of purified RBD, 10× concentration of SYPRO Orange (from 5000× stock solution, ThermoFisher) in 50 mM HEPES, 100 mM NaCl, pH 7.5 and 5% final concentration of DMSO. The small molecules were added to final concentrations of 5 mM. SYPRO Orange fluorescence was monitored over 20-95° C. with a LightCycler® 480 instrument (Roche, USA). The LightCycler® 480 Software (Roche) was used to calculate the first derivate of the resulting melting curve, with the steepest point of the slope being the Tm.

### Target and Ligand Preparation

Neutralization assay using pseudoviral particles. Target cells were infected with single-round luciferase-expressing lentiviral particles as described previously (Prevost et al., 2020). Briefly, 293T cells were transfected by the calcium phosphate method with the lentiviral vector pNL4.3 R-E-Luc (NIH AIDS Reagent Program) and a plasmid encoding for SARS-CoV-2 Spike at a ratio of 5:4. Two days post-transfection, cell supernatants were harvested and stored at −80° C. until use. 293T-ACE2 target cells were seeded at a density of 1×10⁴ cells/well in 96-well luminometer-compatible tissue culture plates (Perkin Elmer) 24 h before infection. Recombinant viruses in a final volume of 100 μl were incubated with the indicated concentrations of small molecules (VE607 and SSAA009E2) up to concentrations of 100 μM for 1 h at 37° C. and were then added to the target cells followed by incubation for 48 h at 37° C.; cells were lysed by the addition of 30 μl of passive lysis buffer (Promega) followed by one freeze-thaw cycle. An LB941 TriStar luminometer (Berthold Technologies) was used to measure the luciferase activity of each well after the addition of 100 μl of luciferin buffer (15 mM MgSO₄, 15 mM KPO₄ [pH 7.8], 1 mM ATP, and 1 mM dithiothreitol) and 50 μl of 1 mM d-luciferin potassium salt (Prolume). The neutralization half-maximal inhibitory dilution (ID₅₀) represents the sera dilution to inhibit 50% of the infection of 293T-ACE2 cells by recombinant viruses.

Microneutralization with authentic virus. One day prior to infection, 2×10⁴ Vero E6 cells were seeded per well in the 96-well flat bottom plate and incubated overnight to permit Vero E6 cell adherence. Compounds dilutions ranged from 0, 0.316, 1, 3.16, 10, 31.6 and 100 μM were performed in a separate 96 well culture plate using DMEM supplemented with penicillin (100 U/mL), streptomycin (100 μg/mL), HEPES, 0.12% sodium bicarbonate, 2% FBS and 0.24% BSA. 10⁴ TCID₅₀/mL of SARS-CoV-2 virus was prepared in DMEM+2% FBS and combined with an equivalent volume of diluted compounds for one hour. After this incubation, all media was removed from the 96 well plate seeded with Vero E6 cells and virus: compounds mixture was added to each respective well at a volume corresponding to 600 TCID₅₀ per well and incubated for one hour further at 37° C. Both virus only and media only (MEM+2% FBS) conditions were included in this assay. All virus-compounds supernatant was removed from wells without disrupting the Vero E6 monolayer. Each diluted compound (100 μL) was added to its respective Vero E6-seeded well in addition to an equivalent volume of MEM+2% FBS and was then incubated for 48 hours. Media was then discarded and replaced with 10% formaldehyde for 24 hours to cross-link Vero E6 monolayer. Formaldehyde was removed from wells and subsequently washed with PBS. Cell monolayers were permeabilized for 15 minutes at room temperature with PBS+0.1% Triton X-100, washed with PBS and then incubated for one hour at room temperature with PBS+3% non-fat milk. An anti-mouse SARS-CoV-2 nucleocapsid protein (Clone 1C7, Bioss Antibodies) primary antibody solution was prepared at 1 μg/mL in PBS+1% non-fat milk and added to all wells for one hour at room temperature. Following extensive washing (3 ×) with PBS, an anti-mouse IgG HRP secondary antibody solution was formulated in PBS+1% non-fat milk. One hour post-room temperature incubation, wells were washed with 3×PBS, substrate (ECL) was added and an LB941 TriStar luminometer (Berthold Technologies) was used to measure the signal each well.

Using the standard calcium phosphate method, 10 μg of Spike expressor and 2.5 μg of a green fluorescent protein (GFP) expressor (pIRES-GFP) were transfected into 2×106 293T cells. 48 h post-transfection, Spike-expressing cells were incubated with 100 μM of VE607 or equivalent volume of vehicle (DMSO) and incubated for 30 min at room temperature. CV3-25 (5 μg/ml) or with sACE2 (100 μg/ml) was added to the cells and incubated for 45 min at 37° C. and sACE2 binding was detected using a polyclonal Goat anti-human ACE2 (RND Systems) at 1/100 dilution at room temperature for 30 min. AlexaFluor-647-conjugated goat anti-human IgG (H+L) Ab (Invitrogen) and AlexaFluor-conjugated donkey anti-goat IgG (H+L) Ab (Invitrogen) was used as secondary antibodies. AF647 and analyzed by flow cytometry. The percentage of transfected cells (GFP+ cells) was determined by gating the living cell population based on viability dye staining (Aqua Vivid, Invitrogen). sACE2-AF594 Binding levels were normalized to signals obtained with the conformationally-independent, anti-S2 CV3-25 mAb (Li et al., 2022; Prevost et al., 2021; Tauzin et al., 2022). Samples were acquired on a LSRII cytometer (BD Biosciences, Mississauga, ON, Canada) and data analysis was performed using FlowJo vX.0.7 (Tree Star, Ashland, OR, USA).

### Chemical Synthesis of Enantiomers of VE607.

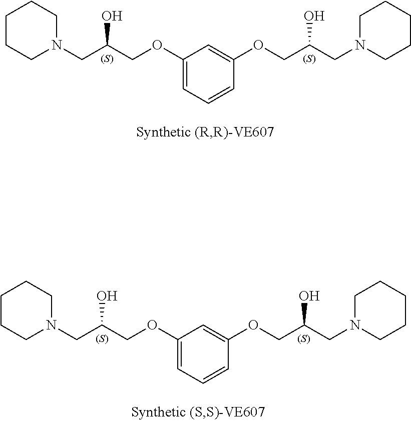

General Procedures. All reactions were conducted in oven-dried glassware under an inert atmosphere of nitrogen, unless otherwise stated. All solvents were reagent or high-performance liquid chromatography (HPLC) grade. Anhydrous THF was obtained from the Pure Solve™ PS-400 system under an argon atmosphere. All reagents were purchased from commercially available sources and used as received. Reactions were magnetically stirred under a nitrogen atmosphere, unless otherwise noted and were monitored by thin layer chromatography (TLC) was performed on pre-coated silica gel 60 F-254 plates (40-55 micron, 230-400 mesh) and visualized by UV light or staining with KMnO₄ and heating. Yields refer to chromatographically and spectroscopically pure compounds. Optical rotations were measured on a JASCO P-200 polarimeter. Proton (¹H) and carbon (¹³C) NMR spectra were recorded on a Bruker Avance III 500-MHz spectrometer. Chemical shifts (6) are reported in parts per million (ppm) relative to chloroform (δ 7.26) or methanol (δ 3.31) for ¹H NMR, and chloroform (δ 77.2) or methanol (δ 49.0). High resolution mass spectra (HRMS) were recorded at the University of Pennsylvania Mass Spectroscopy Service Center on either a VG Micromass 70/70H or VG ZAB-E spectrometer. Lyophilization was performed in a Labconco FreeZone 12 Plus lyophilizer (0.148 mbar). The purity of new compounds were judged by NMR and LCMS (>95%).

### Experimental Procedures

### 1,3-bis(((R)-oxiran-2-yl)methoxy)benzene (+)-2R

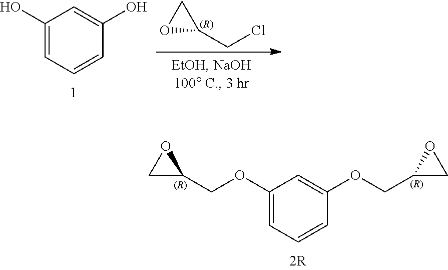

Resorcinol (1) (100 mg, 0.908 mmol, 1 equiv) was dissolved in enantiopure (R)-epichlorohydrin (0.569 mL, 7.27 mmol, 8.0 equiv) and heated to 100° C. A solution of NaOH (72.7 mg, 1.82 mmol, 2.0 equiv) in EtOH (0.75 mL) was then added dropwise. After stirring at 100° C. for three hours, the solution was cooled to r.t., diluted with acetone and filtered through a fritted funnel packed with celite. The filtrate was then concentrated in vacuo which was purified via flash column chromatography (5% to 10% EtOAc/hexanes) to yield the title compound as a yellow oil in 97% yield. ¹H NMR (500 MHz, CDCl₃): δ 7.17 (t, J=8.08 Hz, 1H), 6.55-6.51 (m, 3H), 4.20 (dd, J=7.84, 3.17 Hz, 2H), 3.95 (dd, J=5.66, 5.29 Hz, 2H), 3.36-3.33 (m, 2H), 2.90 (t, J=4.77 Hz, 2H). 2.75 (dd, J=2.63, 2.35 HZ, 2H); ¹³C NMR (125 MHz, CDCl₃): δ 159.76, 130.06, 107.44, 101.94, 68.83, 50.13, 44.87; HRMS (ESI) m/z: [M+Na]⁺ calcd 245.0790, found 245.0791; [α]_{D}²³ +17.28 (c 7.4, MeOH).

### (2R,2′R)-3,3′-(1,3-phenylenebis(oxy))bis(1-(piperidin-1-yl)propan-2-ol) (+)-3RR

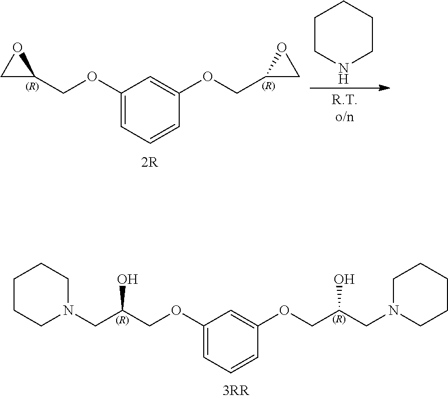

Epoxide 2R (25 mg, 0.113 mmol, 1.0 equiv) was dissolved in neat piperdine (0.177 mL, 1.8 mmol, 16 equiv) at 0° C. and stirred overnight at room temperature. Upon completion, solvent was removed in vacuo, dissolved in DCM, washed with brine, dried with Na₂SO₄, filtered through a cotton plug, and concentrated in vacuo. The crude product mixture was then dissolved in water, frozen, and lyophilized to remove excess piperdine. Product was isolated as a yellow oil in 99% yield. ¹H NMR (500 MHz, CD₃OD): δ 7.17-7.13 (m, 1H), 6.55-6.52 (m, 3H), 4.15-4.10 (m, 2H), 3.96 (dd, J=5.59, 4.31 Hz, 2H), 3.89 (dd, J=5.93, 3.82, 2H), 2.57-2.46 (m, 12H), 1.64-1.59 (m, 8H), 1.51-1.44 (m, 4H); ¹³C NMR (125 MHz, CD₃OD): δ 161.62, 130.96, 108.15, 102.78, 72.15, 68.36, 63.18, 56.28, 47.47, 27.07, 26.77, 25.55, 25.23; HRMS (ESI) m/z: [M+H]⁺ calcd 393.2753, found 393.276; [α]_{D}²³+3.2 (c 3.3, MeOH).

### 1,3-bis(((S)-oxiran-2-yl)methoxy)benzene (−)-2S

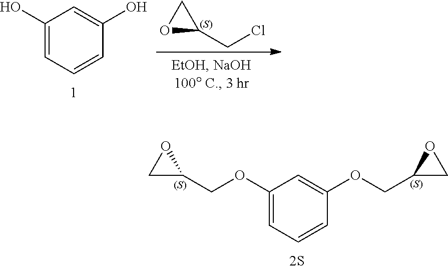

Resorcinol (1) (100 mg, 0.908 mmol, 1 equiv) was dissolved in enantiopure (S)-epichlorohydrin (0.569 mL, 7.27 mmol, 8.0 equiv) and heated to 100° C. A solution of NaOH (72.7 mg, 1.82 mmol, 2.0 equiv) in EtOH (0.75 mL) was then added dropwise. After stirring at 100° C. for three hours, the solution was cooled to r.t., diluted with acetone and filtered through a fritted funnel packed with celite. The filtrate was then concentrated in vacuo which was purified via flash column chromatography (5% to 10% EtOAc/hexanes) to yield the title compound as a yellow oil in 97% yield. ¹H NMR (500 MHz, CDCl₃): δ 7.17 (t, 7.70 Hz, 1H), 6.55-6.51 (m, 3H), 4.20 (dd, J=7.76, 3.13 Hz, 2H), 3.93 (dd, J=5.77, 5.34 Hz, 2H), 3.36-3.33 (m, 2H), 2.90 (t, J=4.87, 2H), 2.75 (dd, 2.68, 2.29, 2H); ¹³C NMR (500 MHz, CDCl₃): δ 159.74, 130.04, 107.42, 101.92, 68.81, 50.11, 44.74; HRMS (ESI) m/z: [M+Na]⁺ calcd 245.0805, found 245.0805; [α]_{D}²³ −17.09 (c 7.4, MeOH).

### (2S,2'S)-3,3′-(1,3-phenylenebis(oxy))bis(1-(piperidin-1-yl)propan-2-ol) (−)-3SS

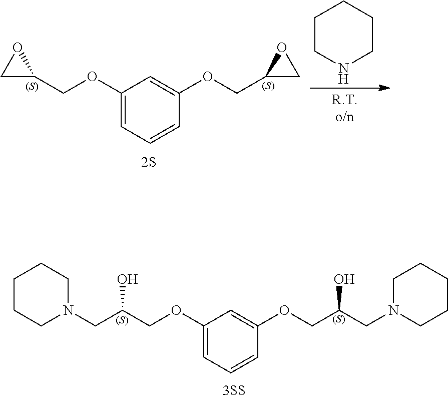

Epoxide 2 (25 mg, 0.113 mmol, 1.0 equiv) was dissolved in neat piperdine (0.177 mL, 1.8 mmol, 16 equiv) at 0° C. and stirred overnight at room temperature. Upon completion, solvent was removed in vacuo, dissolved in DCM, washed with brine, dried with Na₂SO₄, filtered through a cotton plug, and concentrated in vacuo. The crude product mixture was then dissolved in water, frozen, and lyophilized to remove excess piperdine. Product was isolated as a yellow oil in 99% yield. ¹H NMR (500 MHz, CD₃OD): δ 7.18-7.14 (m, 1H), 6.56-6.52 (m, 3H), 4.18-4.14 (m, 2H), 3.96 (dd, J=4.51, 4.45 Hz, 2H), 3.89 (dd, J=5.79, 5.73 Hz), 2.73-2.62 (m, 12H), 1.66-1.57 (m, 8H), 1.56-1.48 (m, 4H); ¹³C NMR (125 MHz, CD₃OD): δ 121.91, 130.96, 108.14, 102.78, 72.15, 68.33, 63.18, 56.27, 47.59, 27.33, 26.78, 25.74, 25.23; HRMS (ESI) m/z: [M+H]⁺ calcd 415.2567, found 415.2567; [α]_{D}²³ −2.96 (c 3.3, MeOH).

### Molecular Modeling

System preparation, modeling, and docking calculation were performed using the Schrödinger Suite package (version 2020-4) [Maestro, Schrödinger, LLC, New York, NY, 2020], using default parameters unless otherwise noted. The target structures were taken from SARS-CoV-1 RBD (PDB ID: 6waq [doi: 10.2210/pdb6WAQ/pdb]) and SARS-CoV-2 RBD (PDB ID: 6w41 [doi: 10.2210/pdb6W41/pdb]) prepared using the Protein Preparation Wizard [doi: 10.1007/s10822-013-9644-8]. To prepare the structures, force field atom types and bond orders were assigned, missing atoms and side-chains were added, protonation states of ionizable amino acid side-chains were determined using PROPKA [doi: 10.1021/ct100578z], water orientations were sampled, and hydrogen bond networks were subsequently optimized by flipping Asn/Gln/His residues and sampling hydroxyl/thiol hydrogen. Constrained energy minimization was then performed using the imperf module from impact [Impact, Schrödinger, LLC, New York, NY, 2020] to generate the structure to be used in the subsequent modeling calculations. Potential binding sites were explored and characterized using the SiteMap tool [doi:10.1021/ci800324m; doi:10.1111/j.1747-0285.2007.00483.x]. VE607 compound was structurally preprocessed using LigPrep [LigPrep, Schrödinger, LLC, New York, NY, 2020] to generate multiple states for stereoisomers, tautomers, ring conformations, and protonation states at a selected pH range. Then, energy minimization was performed with the OPLS3e force field [doi: 10.1021/acs.jctc.8b01026]. The prepared molecular structures were docked into the putative binding sites using Glide [doi: 10.1021/jm030644s; doi: 10.1021/jm0306430] with the standard precision (SP) scoring function to evaluate enrichment of the calculated docked models.

### Quantification and Statistical Analysis.

Statistics were analyzed using GraphPad Prism version 8.0.2 (GraphPad, San Diego, CA, (USA). Every data set was tested for statistical normality and this information was used to apply the appropriate (parametric or nonparametric) statistical test. P values<0.05 were considered significant; significance values are indicated as * p<0.05; ** p<0.01; *** p<0.001; **** p<0.0001.

### Example 2: Differential Scanning Fluorimetry and Docking Suggest that VE607 May Bind the RBD

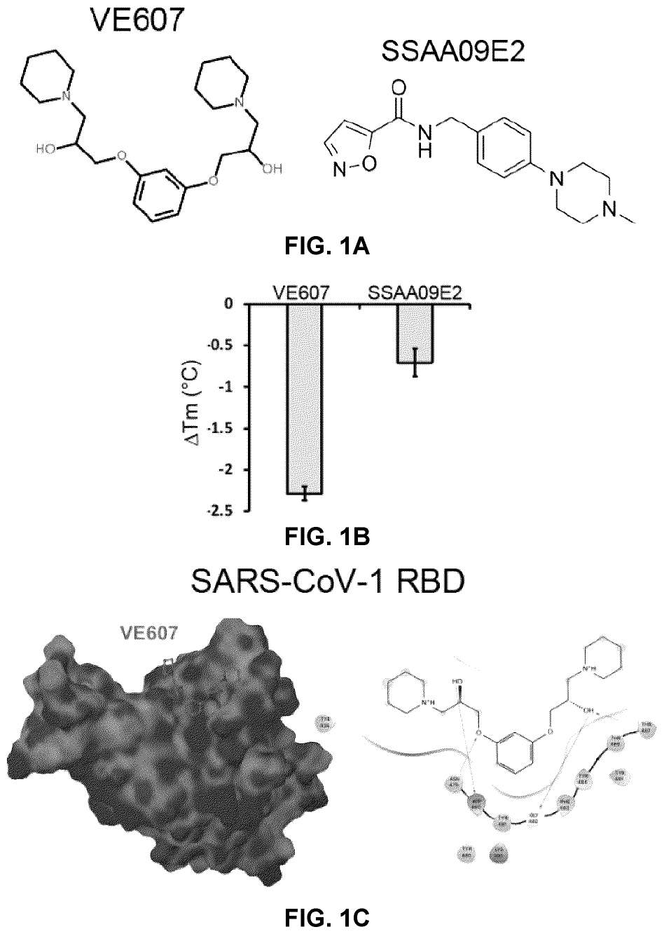

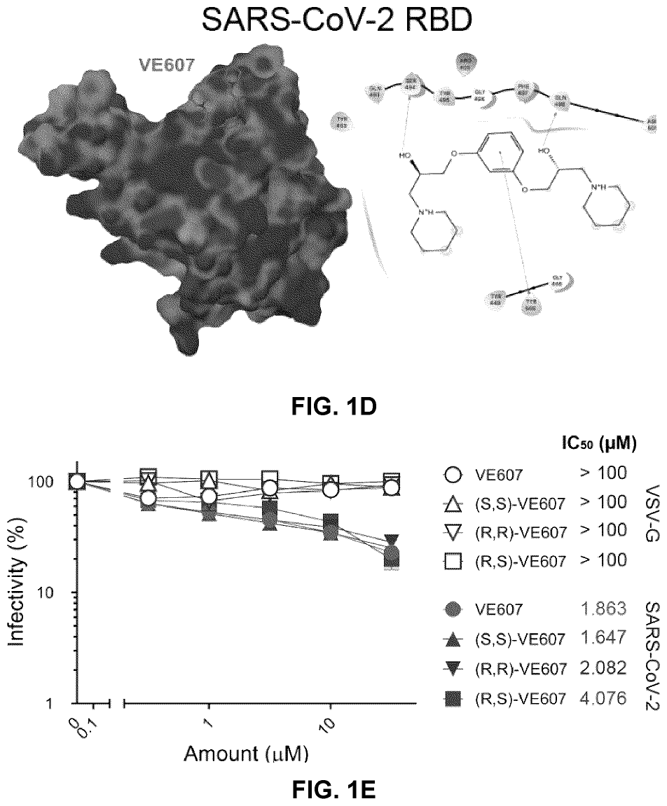

It was initially tested whether the previously described SARS-CoV-1 inhibitors VE607 (Kao et al., 2004) and SSAA09E2 (Adedeji et al., 2013) bind the SARS-CoV-2 RBD (FIG. 1A). Differential scanning fluorimetry (DSF), an approach to identify binding molecules by measuring their effect on the melting of proteins at increasing temperatures (Mashalidis et al., 2013), was used. Incubation with VE607 led to a significant decrease of the melting temperature (ΔTm, −2.3° C.) whereas addition of SSAA09E2 had a smaller, but still measurable effect (ΔTm, −0.7° C.) (FIG. 1B). Since this result suggested binding of VE607 to RBD, in silico docking was next performed using the Glide software, followed by energy minimization. Using this approach, a potential RBD binding site for VE607 at the ACE2 binding interface of SARS-CoV-1 and SARS-CoV-2 RBDs was identified (FIGS. 1C and 1D). It was next tested whether the molecules have effects using in vitro infection assays.

### Example 3: VE607 Inhibits Infection of Pseudoviral Particles and Authentic SARS-CoV-2

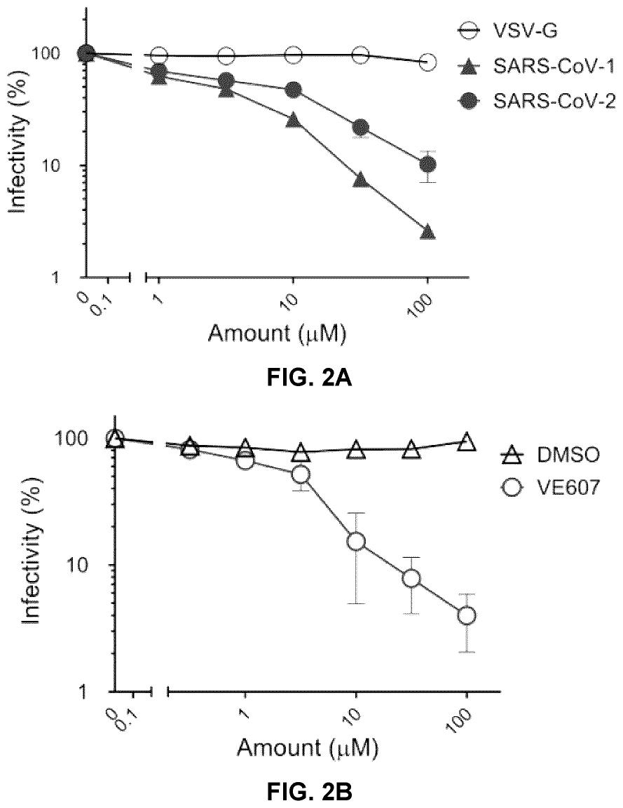

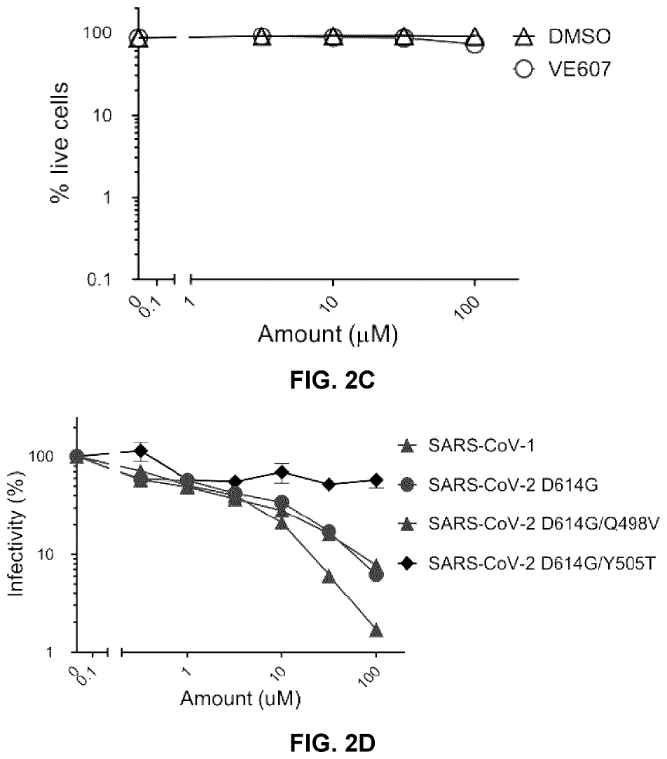

To assess the effects of VE607 and SSAA09E2 using an in vitro model, the SARS-CoV-1 and SARS-CoV-2 S glycoproteins as well as the VSV-G glycoprotein as negative control were expressed on the surface of pseudoviral particles carrying the luciferase gene in their genomes. These particles were used for infection of ACE2-expressing 293T cells (Prevost et al., 2020) in the presence of increasing concentrations of VE607 and SSAA09E2. VE607 specifically inhibited pseudoviral particles bearing the SARS-CoV-1 Spike (IC₅₀=1.47 μM, FIG. 2A). Interestingly, VE607 also inhibited pseudoviral particles expressing the Spike from SARS-CoV-2 (IC₅₀=3.06 μM, FIG. 2A), albeit slightly less efficiently than for SARS-CoV-1. Inhibition was specific since no inhibition of pseudoviral particles bearing the VSV-G was observed (IC₅₀>100 μM, FIG. 2A). To ensure that the inhibitory capacity of VE607 against SARS-CoV-2 was not restricted to pseudoviral particles, it was then evaluated if the inhibitory capacity of VE607 was maintained when using authentic virus. As shown in FIG. 2B, VE607 inhibited authentic SARS-CoV-2 D614G with an IC₅₀ of 2.42 μM. Of note, no toxicity was observed with concentrations up to 100 μM (FIG. 2C). In contrast, SSAA09E2 at concentrations up to 100 M had no effect on the infection of pseudoviral particles.

Commercially available VE607 is a mixture of three stereochemical isomers, comprised of the (S,S)-VE607, (R,R)-VE607, and the meso (R,S)-VE607, mixed at a ratio of 1:1:2. Enantiomers was obtained by chemical synthesis and tested for their ability to neutralize pseudoviral particles bearing the SARS-CoV-2 S protein. No substantial differences in the SARS-CoV-2 pseudoviral inhibition among these enantiomers obtained by synthesis, with the meso (R,S)-VE607 enantiomer being slightly less potent (about 2-fold) than the two other enantiomers (FIG. 1E).

The in silico docking identified RBD residues Y505 and Q498 as potential contact sites for VE607 (FIG. 1D). These residues were mutated in the full-length SARS-CoV-2 D614G Spike and prepared pseudoviral particles to test whether they affected VE607 inhibition. As shown in FIG. 2D, while the Q498V had only a minor effect (IC₅₀=1.80 μM), the Y505T mutant was resistant to VE607 inhibition (IC₅₀>40 μM, FIG. 2D). Alignment of sACE2 on the known ACE2 epitope of the VE607-bound model of RBD displayed significant steric clashes between ACE2 and VE607, suggesting some direct competition for the ACE2 epitope.

### Example 4: VE607 Stabilizes the “Up” Conformation of the S Protein

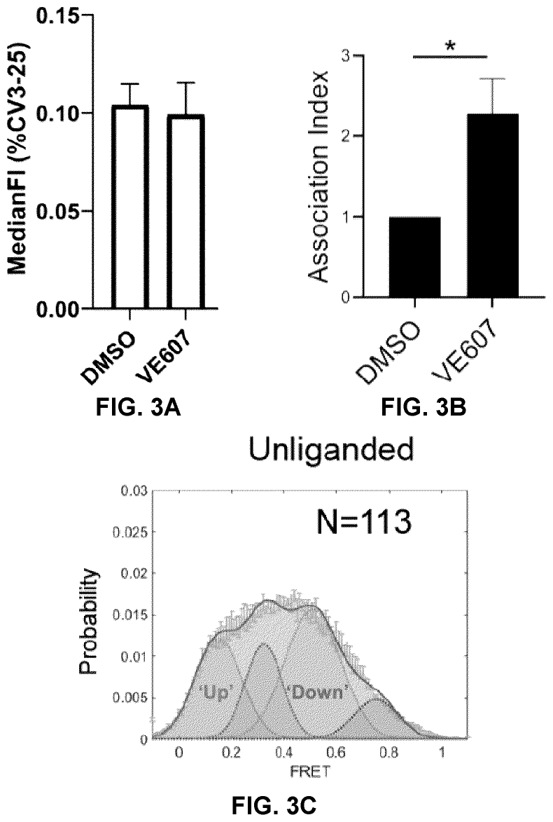

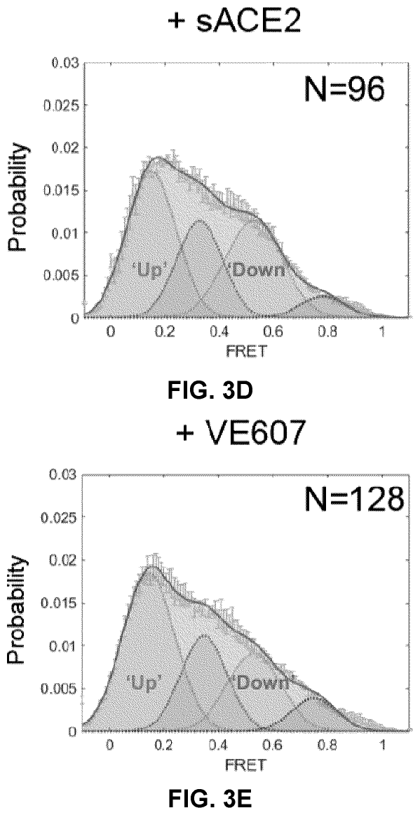

It was next assessed whether VE607 affects the RBD-ACE2 interaction. Briefly, the capacity of VE607 to compete with soluble ACE2 conjugated to Alexa-Fluor 594 for interaction with the full SARS-CoV-2 Spike, expressed at the cell surface, was measured by flow cytometry as described previously (Anand et al., 2020; Anand et al., 2021). Surprisingly, no competition between VE607 (100 μM) and sACE2-AF647 was observed (FIG. 3A). Since the mode of action of some neutralizing antibodies such as CV3-1 involve S1 shedding (Li et al., 2022), it was verified whether VE607 acted in a similar manner. Briefly, Spike expressing 293T cells were radioactive labeled followed by immunoprecipitation of cell lysates and supernatant, as described (Li et al., 2022). In contrast to CV3-1, VE607 decrease shedding, resulting in a more stable trimer (FIG. 3B). To evaluate whether the enhanced stability observed in presence of VE607 altered the conformational landscape of the Spike, single-molecule FRET (smFRET) analysis was performed using viral pseudoparticles carrying modified S glycoproteins labelled with FRET donor and receptor dyes enabling to distinguish the “up” and “down” conformations (Lu et al., 2020). In agreement with previous observations, the unliganded Spike mostly sampled the “down” conformation (FIG. 3C). The addition of sACE2 shifted the conformational landscape of the Spike to the “up” conformation reflecting the receptor bound state (FIG. 3D). Interestingly, and similar to sACE2, it was observed that VE607 also stabilized the “up” conformation (FIG. 3E).

### Example 5: VE607 Inhibits Infection of Some SARS-CoV-2 Variants of Concern

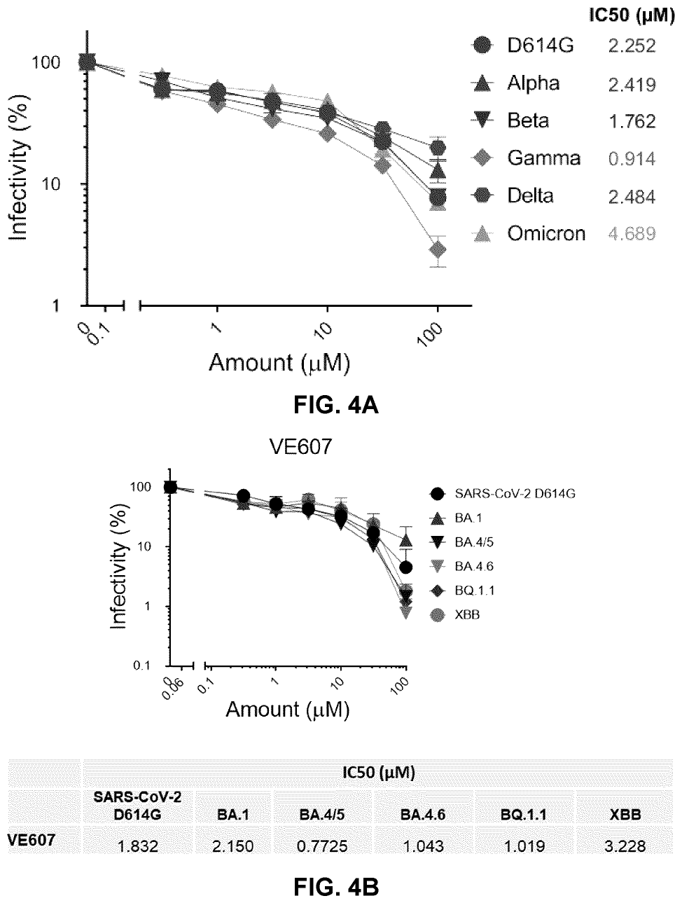

SARS CoV-2 is in constant evolution as variants of concern keep emerging. It was tested whether VE607 inhibits pseudoviral particles bearing the Spike glycoproteins from the major VOCs (Alpha, Beta, Gamma, Delta and Omicron (BA.1, BA.4/5, BA.4.6, XBB or BQ.1.1 sublineage)). VE607 inhibited all VOCs with similar potency with IC₅₀ values in the low micromolar range (FIGS. 4A-B). These results demonstrate that the various amino acid changes in the S-glycoproteins of these variants do not impact the inhibitory potential of VE607.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

Adedeji, A. O., Severson, W., Jonsson, C., Singh, K., Weiss, S. R., and Sarafianos, S. G. (2013). Novel inhibitors of severe acute respiratory syndrome coronavirus entry that act by three distinct mechanisms. J Virol 87, 8017-8028.
Anand, S. P., Chen, Y., Prevost, J., Gasser, R., Beaudoin-Bussieres, G., Abrams, C. F., Pazgier, M., and Finzi, A. (2020). Interaction of Human ACE2 to Membrane-Bound SARS-CoV-1 and SARS-CoV-2 S Glycoproteins. Viruses 12.
Anand, S. P., Prevost, J., Nayrac, M., Beaudoin-Bussieres, G., Benlarbi, M., Gasser, R., Brassard, N., Laumaea, A., Gong, S. Y., Bourassa, C., et al. (2021). Longitudinal analysis of humoral immunity against SARS-CoV-2 Spike in convalescent individuals up to 8 months post-symptom onset. Cell Rep Med 2, 100290.
Ball, P. (2021). The lightning-fast quest for COVID vaccines—and what it means for other diseases. Nature 589, 16-18.
Beaudoin-Bussieres, G., Laumaea, A., Anand, S. P., Prevost, J., Gasser, R., Goyette, G., Medjahed, H., Perreault, J., Tremblay, T., Lewin, A., et al. (2020). Decline of Humoral Responses against SARS-CoV-2 Spike in Convalescent Individuals. mBio 11.
Cai, Y., Zhang, J., Xiao, T., Peng, H., Sterling, S. M., Walsh, R. M., Jr., Rawson, S., Rits-Volloch, S., and Chen, B. (2020). Distinct conformational states of SARS-CoV-2 spike protein. Science 369, 1586-1592.
Chatterjee, D., Tauzin, A., Marchitto, L., Gong, S. Y., Boutin, M., Bourassa, C., Beaudoin-Bussières, G., Bo, Y., Ding, S., Laumaea, A., et al. (2021). SARS-CoV-2 Omicron Spike recognition by plasma from individuals receiving BNT162b2 mRNA vaccination with a 16-weeks interval between doses. 2021.2012.2021.473679.
Cohen, J. (2021). The dream vaccine. Science 372, 227-231.
Dai, W., Zhang, B., Su, H., Li, J., Zhao, Y., Xie, X., Jin, Z., Liu, F., Li, C., Li, Y., et al. (2020). Structure-based design of antiviral drug candidates targeting the SARS-CoV-2 main protease. Science.
Ding, S., Adam, D., Beaudoin-Bussieres, G., Tauzin, A., Gong, S. Y., Gasser, R., Laumaea, A., Anand, S. P., Privé, A., Bourassa, C., et al. (2022). SARS-CoV-2 Spike Expression at the Surface of Infected Primary Human Airway Epithelial Cells. Viruses 14, 5.
Emi, N., Friedmann, T., and Yee, J. K. (1991). Pseudotype formation of murine leukemia virus with the G protein of vesicular stomatitis virus. J Virol 65, 1202-1207.
Gong, S. Y., Chatterjee, D., Richard, J., Prevost, J., Tauzin, A., Gasser, R., Bo, Y., Vezina, D., Goyette, G., Gendron-Lepage, G., et al. (2021). Contribution of single mutations to selected SARS-CoV-2 emerging variants spike antigenicity. Virology 563, 134-145.
Harrison, A. G., Lin, T., and Wang, P. (2020). Mechanisms of SARS-CoV-2 Transmission and Pathogenesis. Trends Immunol 41, 1100-1115.
Hoffmann, M., Kleine-Weber, H., and Pohlmann, S. (2020a). A Multibasic Cleavage Site in the Spike Protein of SARS-CoV-2 Is Essential for Infection of Human Lung Cells. Molecular cell.
Hoffmann, M., Kleine-Weber, H., Schroeder, S., Kruger, N., Herrler, T., Erichsen, S., Schiergens, T. S., Herrler, G., Wu, N. H., Nitsche, A., et al. (2020b). SARS-CoV-2 Cell Entry Depends on ACE2 and TMPRSS2 and Is Blocked by a Clinically Proven Protease Inhibitor. Cell 181, 271-280 e278.
Kao, R. Y., Tsui, W. H., Lee, T. S., Tanner, J. A., Watt, R. M., Huang, J. D., Hu, L., Chen, G., Chen, Z., Zhang, L., et al. (2004). Identification of novel small-molecule inhibitors of severe acute respiratory syndrome-associated coronavirus by chemical genetics. Chem Biol 11, 1293-1299.
Kokic, G., Hillen, H. S., Tegunov, D., Dienemann, C., Seitz, F., Schmitzova, J., Farnung, L., Siewert, A., Hobartner, C., and Cramer, P. (2021). Mechanism of SARS-CoV-2 polymerase stalling by remdesivir. Nat Commun 12, 279.
Krammer, F. (2020). SARS-CoV-2 vaccines in development. Nature 586, 516-527.
Lan, J., Ge, J., Yu, J., Shan, S., Zhou, H., Fan, S., Zhang, Q., Shi, X., Wang, Q., Zhang, L., et al. (2020). Structure of the SARS-CoV-2 spike receptor-binding domain bound to the ACE2 receptor. Nature 581, 215-220.
Li, J., Lai, S., Gao, G. F., and Shi, W. (2021a). The emergence, genomic diversity and global spread of SARS-CoV-2. Nature 600, 408-418.
Li, W., Chen, Y., Prevost, J., Ullah, I., Lu, M., Gong, S. Y., Tauzin, A., Gasser, R., Vezina, D., Anand, S. P., et al. (2021b). Structural Basis and Mode of Action for Two Broadly Neutralizing Antibodies Against SARS-CoV-2 Emerging Variants of Concern. bioRxiv 2021 Aug. 3; 2021.08.02.454546. doi: 10.1101/2021.08.02.454546. Preprint.
Li, W., Chen, Y., Prevost, J., Ullah, I., Lu, M., Gong, S. Y., Tauzin, A., Gasser, R., Vezina, D., Anand, S. P., et al. (2022). Structural basis and mode of action for two broadly neutralizing antibodies against SARS-CoV-2 emerging variants of concern. Cell reports 38, 110210.
Lu, M., Uchil, P. D., Li, W., Zheng, D., Terry, D. S., Gorman, J., Shi, W., Zhang, B., Zhou, T., Ding, S., et al. (2020). Real-Time Conformational Dynamics of SARS-CoV-2 Spikes on Virus Particles. Cell Host Microbe 28, 880-891 e888.
Mannar, D., Saville, J. W., Zhu, X., Srivastava, S. S., Berezuk, A. M., Zhou, S., Tuttle, K. S., Kim, A., Li, W., Dimitrov, D. S., et al. (2021). Structural analysis of receptor binding domain mutations in SARS-CoV-2 variants of concern that modulate ACE2 and antibody binding. Cell Rep 37, 110156.
Mashalidis, E. H., Sledz, P., Lang, S., and Abell, C. (2013). A three-stage biophysical screening cascade for fragment-based drug discovery. Nature protocols 8, 2309-2324.
Nabel, K. G., Clark, S. A., Shankar, S., Pan, J., Clark, L. E., Yang, P., Coscia, A., McKay, L. G. A., Varnum, H. H., Brusic, V., et al. (2021). Structural basis for continued antibody evasion by the SARS-CoV-2 receptor binding domain. Science, eabl6251.
Owen, D. R., Allerton, C. M. N., Anderson, A. S., Aschenbrenner, L., Avery, M., Berritt, S., Boras, B., Cardin, R. D., Carlo, A., Coffman, K. J., et al. (2021). An oral SARS-CoV-2 M(pro) inhibitor clinical candidate for the treatment of COVID-19. Science 374, 1586-1593.
Prevost, J., and Finzi, A. (2021). The great escape?SARS-CoV-2 variants evading neutralizing responses. Cell Host Microbe 29, 322-324.
Prevost, J., Gasser, R., Beaudoin-Bussieres, G., Richard, J., Duerr, R., Laumaea, A., Anand, S. P., Goyette, G., Benlarbi, M., Ding, S., et al. (2020). Cross-Sectional Evaluation of Humoral Responses against SARS-CoV-2 Spike. Cell Rep Med 1, 100126.
Rappazzo, C. G., Tse, L. V., Kaku, C. I., Wrapp, D., Sakharkar, M., Huang, D., Deveau, L. M., Yockachonis, T. J., Herbert, A. S., Battles, M. B., et al. (2021). Broad and potent activity against SARS-like viruses by an engineered human monoclonal antibody. Science 371, 823-829.
Shang, J., Ye, G., Shi, K., Wan, Y., Luo, C., Aihara, H., Geng, Q., Auerbach, A., and Li, F. (2020). Structural basis of receptor recognition by SARS-CoV-2. Nature 581, pages 221-224.
Sharifahmadian, M., Arya, T., Bessette, B., Lecoq, L., Ruediger, E., Omichinski, J. G., and Baron, C. (2017). Monomer-to-dimer transition of Brucella suis type IV secretion system component VirB8 induces conformational changes. The FEBS journal 284, 1218-1232.
Tauzin, A., Gong, S. Y., Beaudoin-Bussieres, G., Vezina, D., Gasser, R., Nault, L., Marchitto, L., Benlarbi, M., Chatterjee, D., Nayrac, M., et al. (2022). Strong humoral immune responses against SARS-CoV-2 Spike after BNT162b2 mRNA vaccination with a 16-week interval between doses. Cell Host Microbe 30, 97-109 e105.
Tauzin, A., Nayrac, M., Benlarbi, M., Gong, S. Y., Gasser, R., Beaudoin-Bussieres, G., Brassard, N., Laumaea, A., Vezina, D., Prevost, J., et al. (2021). A single dose of the SARS-CoV-2 vaccine BNT162b2 elicits Fc-mediated antibody effector functions and T cell responses. Cell Host Microbe 29, 1137-1150 e1136.
Tong, T. (2009). Therapies for coronaviruses. Part I of II—viral entry inhibitors. Expert Opinion on Therapeutic Patents 19.
Ullah, I., Prevost, J., Ladinsky, M. S., Stone, H., Lu, M., Anand, S. P., Beaudoin-Bussieres, G., Symmes, K., Benlarbi, M., Ding, S., et al. (2021). Live imaging of SARS-CoV-2 infection in mice reveals that neutralizing antibodies require Fc function for optimal efficacy. Immunity 54, 2143-2158 e2115.
Wang, G., Yang, M. L., Duan, Z. L., Liu, F. L., Jin, L., Long, C. B., Zhang, M., Tang, X. P., Xu, L., Li, Y. C., et al. (2021). Dalbavancin binds ACE2 to block its interaction with SARS-CoV-2 spike protein and is effective in inhibiting SARS-CoV-2 infection in animal models. Cell Res 31, 17-24.
Wrapp, D., Wang, N., Corbett, K. S., Goldsmith, J. A., Hsieh, C. L., Abiona, O., Graham, B. S., and McLellan, J. S. (2020). Cryo-EM structure of the 2019-nCoV spike in the prefusion conformation. Science 367, 1260-1263.
Wu, F., Zhao, S., Yu, B., Chen, Y. M., Wang, W., Song, Z. G., Hu, Y., Tao, Z. W., Tian, J. H., Pei, Y. Y., et al. (2020). A new coronavirus associated with human respiratory disease in China. Nature 579, 265-269.
Yan, R., Zhang, Y., Li, Y., Xia, L., Guo, Y., and Zhou, Q. (2020). Structural basis for the recognition of SARS-CoV-2 by full-length human ACE2. Science 367, 1444-1448.
Yang, H., and Rao, Z. (2021). Structural biology of SARS-CoV-2 and implications for therapeutic development. Nature reviews Microbiology 19, 685-700.
Yang, Z., Han, Y., Ding, S., Finzi, A., Mothes, W., and Lu, M. (2021). SARS-CoV-2 variants exhibit increased kinetic stability of open spike conformations as an evolutionary strategy. 2021.2010.2011.463956.
Yin, W., Luan, X., Li, Z., Zhou, Z., Wang, Q., Gao, M., Wang, X., Zhou, F., Shi, J., You, E., et al. (2021). Structural basis for inhibition of the SARS-CoV-2 RNA polymerase by suramin. Nat Struct Mol Biol 28, 319-325.
Zhang, L., Lin, D., Sun, X., Curth, U., Drosten, C., Sauerhering, L., Becker, S., Rox, K., and Hilgenfeld, R. (2020). Crystal structure of SARS-CoV-2 main protease provides a basis for design of improved alpha-ketoamide inhibitors. Science 368, 409-412.
Zhu, N., Zhang, D., Wang, W., Li, X., Yang, B., Song, J., Zhao, X., Huang, B., Shi, W., Lu, R., et al. (2020a). A Novel Coronavirus from Patients with Pneumonia in China, 2019. N Engl J Med 382, 727-733.
Zhu, Z. L., Qiu, X. D., Wu, S., Liu, Y. T., Zhao, T., Sun, Z. H., Li, Z. R., and Shan, G. Z. (2020b). Blocking Effect of Demethylzeylasteral on the Interaction between Human ACE2 Protein and SARS-CoV-2 RBD Protein Discovered Using SPR Technology. Molecules 26.

1. A method for preventing or treating a SARS-CoV-2 infection or a related disease and/or for reducing the risk of developing a SARS-CoV-2-related disease or the severity of a SARS-CoV-2-related disease in a subject in need thereof, the method comprising administering to the subject an effective amount of 3,3′-(1,3-phenylenebis(oxy))bis(1-(piperidin-1-yl)propan-2-ol (VE607) or a pharmaceutically acceptable salt thereof.

2. The method of claim 1, wherein the method is for reducing the risk of developing a SARS-CoV-2-related disease or the severity of a SARS-CoV-2-related disease in a subject.

3. A method for blocking the entry of SARS-CoV-2 in an ACE2-expressing cell, the method comprising contacting the SARS-CoV-2 and/or cell with an effective amount of 3,3′-(1,3-phenylenebis(oxy))bis(1-(piperidin-1-yl)propan-2-ol (VE607) or a pharmaceutically acceptable salt thereof.

4. The method of claim 1, wherein the SARS-CoV-2 is a SARS-CoV-2 variant.

5. The method of claim 4, wherein the SARS-CoV-2 variant comprises one or more of the following mutations in the Spike protein: L5F, S13I, L18F, T19R, T20N, P26S, A67V, del69-70, G75V, T76I, D80Y, D80A, T95I, S98F, R102I, D138Y, G142D, del142-144, del144, W152C, E154K, EFR156-158G, F157L, R190S, ins214EPE, D215G, A222V, del246-252, D253G, W258L, N354D, F342L, V367F, K417N, K417T, A435S, W436R, N439K, N440K, G446V, L452R, Y453F, K458R, G476S, S477N, S477G, T478K, V483A, E484K, E484Q, F490S, N501Y, N501S, N501T, A570D, Q613H, D614G, A626S, A653V, H655Y, Q677H, Q677P, P681H, P681R, A701V, T716I, D796H, D796Y, T859N, F888L, D950N, S982A, T1027I, Q1071H, E1092K, H1101Y, D1118H, V1176F, G1219V, and V1122L.

6. The method of claim 4, wherein the SARS-CoV-2 variant is the Alpha, Beta, Gamma, Delta or Omicron variant.

7. The method of claim 1, wherein the VE607 or pharmaceutically acceptable salt thereof is present in a pharmaceutical composition comprising a pharmaceutically acceptable carrier or excipient.

8. The method of claim 7, wherein the pharmaceutical composition is an oral composition.

9. The method of claim 8, wherein the pharmaceutical composition is a tablet or a capsule.

10. The method of claim 1, wherein the VE607 or pharmaceutically acceptable salt thereof is administered in combination with an additional active agent.

11. The method of claim 10, wherein the additional active agent is a pain killer, an anti-nausea agent, an anti-coagulant, an anti-inflammatory agent, an immunotherapeutic agent, a SARS-CoV-2 vaccine or a SARS-CoV-2 inhibitor.

12. The method of claim 1, wherein the subject is an immunosuppressed or immunocompromised subject.

13-36. (canceled)


SEQUENCE LISTING (1 sequence)

SEQ ID NO: 1 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT